SEQUENCE LISTING (16 sequences)

SEQ ID NO: 1 - DNA, 22 bp, Artificial Sequence
  synthetic oligonucleotide
cgtggatgcagaacttgacaac

SEQ ID NO: 2 - DNA, 25 bp, Artificial Sequence
  synthetic oligonucleotide
cacacagtaagcccaggtagtaaaa

SEQ ID NO: 3 - DNA, 20 bp, Artificial Sequence
  synthetic oligonucleotide
gatggcttcagggctaaaca

SEQ ID NO: 4 - DNA, 19 bp, Artificial Sequence
  synthetic oligonucleotide
cagcaggtgggcaaggtat

SEQ ID NO: 5 - DNA, 17 bp, Artificial Sequence
  synthetic oligonucleotide
tggagacggcgatggag

SEQ ID NO: 6 - DNA, 20 bp, Artificial Sequence
  synthetic oligonucleotide
caggcttggaccgctactct

SEQ ID NO: 7 - DNA, 25 bp, Artificial Sequence
  synthetic oligonucleotide
acgcctctgtcatgtaccaaatcct

SEQ ID NO: 8 - DNA, 23 bp, Artificial Sequence
  synthetic oligonucleotide
ggcctcgacgatgaagctgtatt

SEQ ID NO: 9 - DNA, 25 bp, Artificial Sequence
  synthetic oligonucleotide
caaatgtgaagcggtcaacaaagtc

SEQ ID NO: 10 - DNA, 25 bp, Artificial Sequence
  synthetic oligonucleotide
atgctttccccaatacttgtcgtct

SEQ ID NO: 11 - DNA, 20 bp, Artificial Sequence
  synthetic oligonucleotide
ggaaggggacaaatataagc

SEQ ID NO: 12 - DNA, 19 bp, Artificial Sequence
  synthetic oligonucleotide
ggcaaggatgggtacatag

SEQ ID NO: 13 - DNA, 21 bp, Artificial Sequence
  synthetic oligonucleotide
accctaaggccaaccgtgaaa

SEQ ID NO: 14 - DNA, 25 bp, Artificial Sequence
  synthetic oligonucleotide
tcattgccgatagtgatgacctgac

SEQ ID NO: 15 - DNA, 20 bp, Artificial Sequence
  synthetic oligonucleotide
ctgatgcccccatgttcgtc

SEQ ID NO: 16 - DNA, 25 bp, Artificial Sequence
  synthetic oligonucleotide
caccctgttgctgtagccaaattcg

(19) United States
(12) Patent Application Publication
(10) Pub. No.: US 2007/0274966 A1
(43) Pub. Date: Nov. 29, 2007

(54) Neural regeneration with transplanted AC133 positive cells

(76) Inventors: Masakazu Ishikawa, Hiroshima (JP); Mitsuo Ochi, Hiroshima (JP); Takayuki Asahara, Kobe-shi (JP)

Correspondence Address: KRATZ, QUINTOS & HANSON, LLP, 1420 K Street, N.W., Suite 400, WASHINGTON, DC, 20005, US

(21) Appl. No.: 11/711,898

(22) Filed: Feb. 28, 2007

(30) Foreign Application Priority Data
Apr. 18, 2006 (JP) 2006-114075

(51) Int. Cl. A61K 35/12; A61P 25/00; A61P 25/02

(52) U.S. Cl. 424/93.7

(57) ABSTRACT

We focused attention on AC133 positive cells as a cell source. AC133 positive cells were transplanted to an injured sciatic nerve model and an injured spinal cord model and were found to have very intensive neural regeneration ability. Such AC133 positive cells are easily available from the peripheral blood with reduced burden on a donor. In addition, they are free from ethical questions and are found to be used as a very useful cell source in neural regenerative treatments with transplanted cells. According to this invention, there is provided a cell source for neural regenerative treatments with transplanted cells, which is more easily available and has a higher neural regeneration ability than equivalents.

### BACKGROUND OF THE INVENTION

1. Technical Field

The present invention relates to neural regenerative treatments with transplanted AC133 positive cells derived from peripheral blood.

2. Description of the Related Art

Nerves are tissues that play very important roles in vital activities of all animals. It is difficult, however, to remedy patients with a nerve injury completely according to current medical techniques, because the nerve tissues are inferior in regenerative capacity to other body tissues. Various investigations have therefore been made to therapeutic methods for regenerating injured neural tissues. Initially, attempts have been made to provide treatments with biological substances such as silicone tubes, but such treatments did not yield sufficient efficacies and failed to be established as efficacious therapeutic methods.

Under such circumstances, rapidly advanced cytobiology has revealed the presence of neural stem cells that are capable of differentiating into various cells belonging to the nervous system. This has induced attentions on a novel therapeutic approach, i.e., neural regeneration with transplanted cells. Main streams in current studies for neural regenerative treatments based on cell transplantation in orthopedics are treatments with neural stem cells (Murakami T. et al., Brain Res., 2003, Fujiwara Y. et al., Neurosci Lett., 2004) and mesenchymal stem cells derived from a bone marrow (Neuhuber B. et al., Brain Res., 2005).

On the other hand, another approach has been made on neural regeneration through angiogenesis, because interactions between the nerve and blood vessels in neurogenesis and axon guidance have been revealed. For example, Taguchi A. et al. reported that CD34 positive cells derived from cord blood were transplanted to a mouse cerebral infarction model, and this led to neural regeneration through angiogenesis (J Clin Invest., 2004).

AC133 is a glycoprotein antigen having a molecular weight of 120 kDa (Yin H. et al., Blood, 1997). The AC133 antigen is known to be expressed selectively on surfaces of CD34 positive haematopoietic stem cells/progenitor cells derived from human fetal liver, bone marrow, and blood. All non-committed CD34 positive cells and CD34 positive cells committed on the granulocyte/mononuclear leukocyte pathway are dyed with the AC133 antibody. However, human umbilical vein endothelial cells and fibroblasts are not dyed with the AC133 antibody, although they are dyed with the CD34 antibody. Accordingly, AC133 is used as a marker for immature/undeveloped cell populations. For example, PCT Japanese Translation Patent Publication No. 2005-526482 describes that, when stem cells are separated typically from the cord blood, a bone marrow, or the peripheral blood using the VEGFR-1 antibody, the target cells are separated with AC133 (or CD34) as a marker as pretreatment or posttreatment.

### SUMMARY OF THE INVENTION

A bottleneck of neural regenerative treatments with transplanted cells resides in properties of cells used in the treatment. The above-mentioned neural stem cells include ethical questions currently in Japan and are difficult to be practically clinically applied. The bone marrow-derived mesenchymal stem cells must be cultured and thereby require facilities and cause increased cost, although they are relatively easily collected and are available as autologous cells.

The cord blood-derived CD34 positive cells are available without problems as mentioned above, but they are expected to fail to yield clinically sufficient therapeutic efficacy especially on a highly injured nerve. This is because populations selected as CD34 positive cells probably contain much of differentiated cells.

Accordingly, there are still demands for searching cell sources to provide neural regenerative treatments with transplanted cells.

The present inventors focused attention on AC133 positive cells as a cell source for neural regenerative treatments. Such AC133 positive cells belong to a population more immature than CD34 positive cells, are found to have high proliferation potency, and are separable from the peripheral blood which is considered to be clinically applied easily. The present inventors have made investigations on the potency of the AC133 positive cells in experimental systems of an in vitro organ culture model, an in vivo injured peripheral nerve model, and an injured spinal cord model, and have found that the cells intensively promote neural regeneration in all the experimental systems. The present invention has been made based on these findings.

Specifically, the present invention relates to therapeutic agents as follows:

1. A therapeutic agent for a neuropathy selected from the group consisting of a spinal cord injury, a sciatic nerve injury, and a neurotmesis, including AC133 positive cells.

2. The therapeutic agent according to (1), in which the AC133 positive cells are derived from peripheral blood, cord blood, placental blood, or a bone marrow.

3. The therapeutic agent according to (2), in which the AC133 positive cells are derived from peripheral blood.

4. The therapeutic agent according to (3), in which the AC133 positive cells are of human origin.

5. The therapeutic agent according to any one of (2) to (4), in which the neuropathy is a spinal cord injury.

6. The therapeutic agent according to any one of (2) to (4), in which the neuropathy is a sciatic nerve injury.

7. The therapeutic agent according to any one of (2) to (4), in which the neuropathy is a neurotmesis.

8. The therapeutic agent according to (6), in which the sciatic nerve injury is an entrapment neuropathy.

The present invention also relates to treatment methods as follows:

9. A method of treating a neuropathy selected from the group consisting of a spinal cord injury, a sciatic nerve injury, and a neurotmesis, including the step of transplanting AC133 positive cells.

10. The method according to (9), in which the AC133 positive cells are derived from peripheral blood, cord blood, placental blood, or a bone marrow.

11. The method according to (10), in which the AC133 positive cells are derived from peripheral blood.

12. The method according to (11), in which the AC133 positive cells are of human origin.

13. The method according to any one of (10) to (12), in which the neuropathy is a spinal cord injury.

14. The method according to any one of (10) to (12), in which the neuropathy is a sciatic nerve injury.

15. The method according to any one of (10) to (12), in which the neuropathy is a neurotmesis.

16. The method according to (15), in which the sciatic nerve injury is an entrapment neuropathy.

Initially, AC133 positive cells are capable of intensively promoting neural regeneration. The mechanism thereof is probably as follows. The AC133 positive cells belong to a fraction including vascular endothelial progenitor cells and thereby may act to promote neural regeneration through revascularization. In addition, transplanted AC133 positive cells will secrete various factors, such as vascular endothelial growth factor (VEGF), which may act as factors for improving the environment in neural regeneration. Secondarily, these cell groups generally circulate in the peripheral blood, can be relatively easily separated with a device generally clinically used, and are available as autogenic cells. The treatment with these cells can therefore be a transplantation treatment that is easily clinically applied, in contrast to neural stem cells which cause many ethical questions.

### BRIEF DESCRIPTION OF THE DRAWINGS

FIG. 1 is a photograph of a model of in vitro organ culture in brain cortex and spinal cord used in Example 1, in which the cap of a mushroom-like image indicates the cortical tissue, and the stem indicates the spinal cord tissue. In this experiment, AC133 positive cells were dropped to the regions indicated by the arrows.

FIG. 2 shows visualized images of axonal growth with AC133 positive cell group labeled with a fluorescent dye Dil in the in vitro organ culture model of FIG. 1. Mild axonal growth was observed in an MNC group, and significant axonal growth was observed in an AC133 positive cell group.

FIG. 3 is a graph showing the comparison of numbers of axons protruding 500, 1000, 1500, 2000 and 2500 μm or more from the origin (the junction between the cortex and the spinal cord), with the abscissa indicating the distance from the origin (μm) and the ordinate indicating the number of axons protruding over the distance. In the AC133 positive cell group, a large number of axons protruded to a further extent.

FIG. 4 shows photographs illustrating the procedures in Example 2, in which FIGS. 4(A), 4(B), and 4(C) show exposure of the sciatic nerve, dissection of the sciatic nerve, and bridging with a silicone tube containing AC133 positive cell group, respectively.

FIG. 5 shows photographs of dissected bridged portion in week 8 after transplantation, of a control group (A) and an AC133 positive cell group (B), respectively. Continuity of the nerve was observed only in the AC133 positive cell group. The muscular evoked potential (D) of the AC133 positive cell group equivalent to the potential of normal sciatic nerve (C) could be led. These results demonstrate that the injured part of sciatic nerve is physically and functionally regenerated.

FIG. 6 illustrates photographs showing the procedures in Example 3, i.e., laminectomy (A), exposure of spinal cord (B), and contusion formation on exposed spinal cord (C).

FIG. 7 shows a photomicrograph and immunostaining of an injured area one day after the transplantation of AC133 positive cell group to an injured spinal cord model in Example 3. Blue, red, and green regions are colored regions as a result of DAPI staining, human nuclear antigen staining, and isolectin B4 staining, respectively. These results verify that intravenously administered AC133 positive cells accumulate and differentiate into vascular endothelial cells in the injured area.

FIG. 8 shows photomicrographs of the injured area three weeks after the transplantation, in which a PBS group (A) showed a cavity in the injured spinal cord, but the AC133 positive cell group (B) did not show a cavity.

FIG. 9 is a graph showing Basso-Beattie-Bresnahan Locomotor (BBB) scores of the AC133 positive cell group (violet) and PBS group (blue) six weeks after the transplantation. A significant improvement was observed as a result of the transplantation of the AC133 positive cell group.

FIG. 10 shows the toluidine blue stains (A) of regenerated tissues derived from the AC133 positive cell-transplanted group and the control group, and the statistical analyses (B) at four points of assessment. In FIG. 10(B), the upper-left graph, upper-right graph, lower-left graph, and lower-right graph show the number of myelinated fibers, the axon diameter, the myelin thickness, and the percentage of the neural tissues, respectively.

FIG. 11 shows the reverse transcriptase polymerase chain reaction (RT-PCR) analyses of regenerated tissues derived from the AC133 positive cell group eight week after the transplantation.

### BEST MODE FOR CARRYING OUT THE INVENTION

As is described above, the present invention uses AC133 positive cells in a neural regenerative treatment.

Such AC133 positive cells can be collected typically from peripheral blood, cord blood, placental blood, or a bone marrow. In practical clinical application, the peripheral blood is advantageously used as a source in view typically of easy availability and light burden on a donor.

By using the peripheral blood, advantages of the present invention can be enjoyed maximally. It should be noted, however, a feature of the present invention resides in the use of AC133 positive cells in a neural regenerative treatment, and the source of AC133 positive cells is not limitative at all.

Examples of a process for separating an AC133 positive cell group from the source thereof, such as the peripheral blood, include magnetic cell sorting (MACS) and fluorescence activated cell sorting (FACS) known by those skilled in the art. Reagents, apparatuses, and other devices necessary for carrying out MACS and FACS are available, for example, from Miltenyi Biotech GmbH and Becton, Dickinson and Company, respectively. As for details, refer to these companies.

AC133 positive cells may be derived from any of autologous cells (auto), allo-cells (allo), and xenogeneic cells (zeno). When the present invention is applied to a treatment of a human patient, the cells are preferably autologous cells or allo-cells, and most preferably autologous cells. Examples of xenogeneic sources include mammals such as pigs and monkeys. Even xenogeneic cells, however, can be sufficiently possibly transplanted and survival in the nerve systems using an acceptable level of immunosuppressive drug, because the nerve systems, especially the central nervous system, are more resistant to the occurrence of immunorejection than other organs and tissues. Examples of currently used immunosuppressive drugs include cyclosporin, acrolimus hydrate, cyclophosphamide, azathioprine, mizoribine, and methotrexate.

AC133 positive cells collected from any of the sources, such as the peripheral blood of a patient himself or a donor, can be directly transplanted, or transplanted after once being cultured and proliferated in vitro.

AC133 positive cells can be collected as a cell suspension and can be transplanted in various forms. The transplantation may be carried out, for example, by injecting a cell suspension locally to an injured nerve area, a nerve suture area or myelocele, by embedding a cell suspension in a carrier such as an atelocollagen gel which shows no antigenicity, and injecting the resulting carrier to the target area, or by preparing a hybrid of cells with an artificial nerve and transplanting the hybrid. When cells are transplanted to an injured spinal cord area, cells may be transplanted by carrying out surgical laminectomy to expose the spinal cord and injecting the cells. Alternatively, cells may be injected to the injured area with minimal invasion without laminectomy while monitoring the area upon an MRI image. In addition, AC133 positive cells can be delivered to an injured area even through transvenous injection, as shown in examples mentioned later. It should be noted, however, any transplantation procedure is included within the scope of the present invention, as long as the procedure uses AC133 positive cells in a neural regenerative treatment. This is because a feature of the present invention resides in the use of AC133 positive cells in a neural regenerative treatment, and the transplantation procedure of AC133 positive cells to an injured area is not an essential component of the present invention.

AC133 positive cells can be combined with an arbitrary pharmaceutically acceptable carrier upon transplantation, as long as not adversely affecting the efficacy thereof.

Examples of such pharmaceutically acceptable carriers include water, physiological saline, phosphate-buffered physiological saline, dextrose, glycerol, ethanol, and atelocollagen used in the after-mentioned examples. The arbitrary pharmaceutically acceptable carrier may further include a small amount of an adjuvant or auxiliary known in the art. AC133 positive cells for use in the present invention can be formulated so as to provide rapid release, sustained release, or delayed release, using a technique known in the art. AC133 positive cells can exhibit sufficient efficacy even when used as a single component for a neural regenerative treatment, but they may be used in combination with one or more other efficacious ingredients according to circumstances. Even when used in combination with one or more other ingredients, AC133 positive cells exhibit efficacy equal to or higher than those described in the present specification. Thus, it should be understood that such an embodiment is also included within embodiments according to the present invention.

Examples of targets to which a treatment according to the present invention is applied include entrapment neuropathy of peripheral nerve, neurotmesis, defect, and spinal cord injury due typically to trauma. The regenerative treatment according to the present invention has been verified to be effective for not only partial neurotmesis but also complete neurotmesis, in the after-mentioned examples. Regarding to spinal cord injury, the treatment can be applied to any area including an area proximal to the brain, such as medulla oblongata and cervical cord, and an area distal to the brain, such as thoracic cord, lumbar spinal cord, and sacral cord. The application is not specifically limited by the degree of seriousness of symptom, and the treatment can be applied to symptoms ranging from mild paralysis to severe symptoms with paraplegia, tetraplegia, or respiratory paralysis. The cause of injury is not specifically limited, and the treatment can be applied to a wide variety of injuries including traumatic injuries such as those caused by traffic accidents or falling accidents, as well as injuries caused by diseases, such as scission of pyramidal system caused by cerebral stroke.

A treatment according to the present invention is preferably applied in an acute stage, i.e., within several hours after being injured, but it may be applied in a chronic stage, for example, several years after being injured.

Terms used for illustrating the present invention are used in the same meanings as generally recognized in the art. Some of these terms specifically necessary for clearly illustrating the scope of the present invention are defined as follows.

The term “AC133 positive cells” refers to cells expressing surface antigen AC133 (also referred to as “CD133”).

The term “peripheral blood” refers to blood circulating in systemic blood vessels.

The term “peripheral blood-derived AC133 positive cells” means and includes not only AC133 positive cells collected from the peripheral blood, but also AC133 positive cells which have been cultured and grown in vitro after being collected from the peripheral blood. The terms “cord blood-derived AC133 positive cells”, “bone marrow-derived AC133 positive cells”, and “placental blood-derived AC133 positive cells” are as with above.

The term “neurotmesis” means and includes not only complete laceration but also partial laceration of a nervous system tissue.

The present invention will be illustrated in further detail with reference to several examples below, which, however, by no means limit the scope of the present invention.

### EXAMPLE 1

The axonal growth promotion ability of AC133 positive cells derived from human peripheral blood was investigated using an in vitro organ culture model in this example.

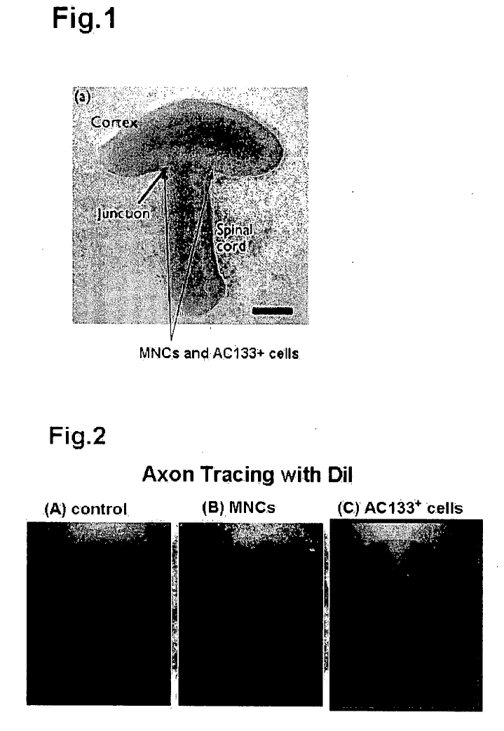

An in vitro organ culture model of brain cortex and spinal cord was prepared according to the method reported previously (Oishi Y et al., J Neurotrauma 21:339-356(2004)). Brains and spinal cords were collected from Sprague-Dawley (SD) rats on postnatal day 3 (P3) or day 7 (P7). The brains were sectioned using a Vibratome (Dosaka EM). The region of cortex was dissected from the coronal sections, and the spinal cord was bisected in the sagittal plane. The dissected cortex and spinal cord were placed on membranes (Millicell-CM; Millipore, Billerica, Mass., USA) in the 1 ml of serum-based medium (50% basal medium Eagle with Earle's Salts (BME; Sigma), 25% inactivated horse serum (Gibco), 25% Earle's Balanced Salt Solution (EBSS; Sigma), 1 mM L-glutamine and 0.5% D-glucose) in 6-well tissue culture plates. The cortex and the spinal cord were incubated for 1 day, then, on the second day, the spinal cord pieces were aligned adjacent to the white matter of the cortex (FIG. 1). The co-cultures were incubated in an atmosphere with 5% CO₂ at 37° C. The medium was replaced every 3 days. The co-cultures were incubated for 14 days.

AC133 positive cells (1×10⁴ in 2 μl phosphate buffered saline (PBS)) were dropped on the spinal cord tissue just after the cortical tissue and the spinal cord tissue contacted each other (AC133 positive cell group). The AC133 positive cells had been obtained by collecting mononuclear leukocyte fractions from the peripheral blood by density gradient centrifugation using a Histopaque-1077 (Sigma), and separating AC133 positive cells from the fractions using an automatic magnetic cell separator autoMACS™ (Miltenyi Biotec) and a CD133 Cell Isolation Kit.

For comparison, mononuclear cells (MNC; 1×10⁴ in 2 μl PBS) and PBS alone, respectively, instead of AC133 positive cells, were dropped in the same manner to yield control groups (MNC group and PBS group, respectively).

Axon projections from the cortex to the spinal cord were labeled by anterograde axon staining with a fluorescent dye Dil. Specifically, the co-cultures were fixed in 4% paraformaldehyde at 4° C. for 5 days. Crystals of Dil were placed in the center of cortex, and the co-cultures were incubated in 0.1 M phosphate buffer at 37° C. in an atmosphere with 5% CO₂ for further 14 days. The results are shown in FIG. 2. The MNC group showed mild axonal growth, but the AC133 positive cell group showed significant axonal growth.

To analyze axonal growth, the number of axons passing through reference lines running parallel to the junction between the cortex and the spinal cord 500, 1000, 1500, 2000 and 2500 μm from the junction was counted. The results are expressed as mean ± s.e. The statistical significance of differences in parameters was assessed by the Mann-Whitney U test.

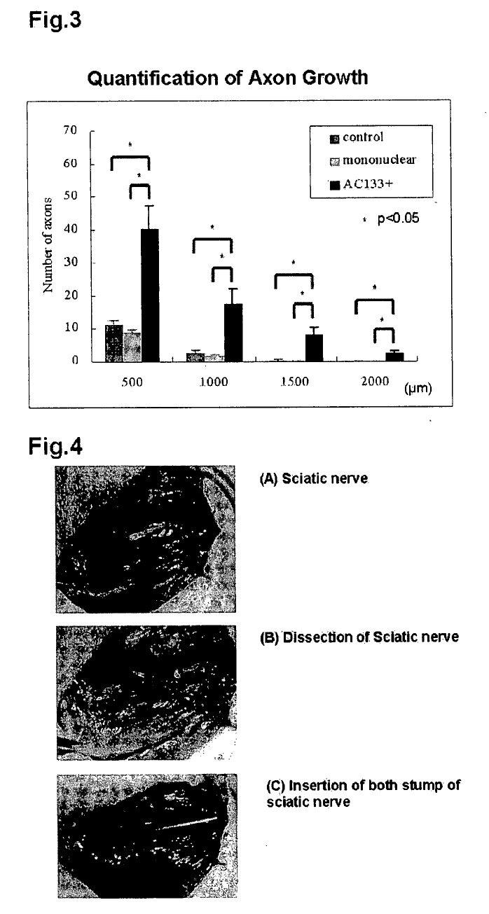

The results are shown in FIG. 3. In the regions 500 and 1000 μm from the junction, some axons were detectable even in the MNC group and the PBS group, but the number of projected axons was further larger in the AC133 positive cell group than in these groups. In the regions 1500 and 2000 μm from the junction, some axons in the AC133 positive cell group projected and reached these regions, but substantially no axons in the MNC group and the PBS group did.

The projection (length), and amount of axons are factors largely participating in regeneration of functions. If axons project to small lengths, even when projected, they are merely ectopic projections which do not reach a target area, and they provide only small functional regeneration. To regenerate injured nerves in the true meaning, axons must be formed in a sufficient amount and have sufficient lengths to reconstruct projections equivalent to normal projections.

Accordingly, these results indicate that transplanted AC133 positive cells have such an intensive axonal growth promotion ability as to recover functions of injured nerves.

### EXAMPLE 2

The regeneration efficacy on peripheral nerve of transplanted human peripheral blood-derived AC133 positive cells in an immunodeficient rat sciatic nerve defect model was studied herein.

Nude rats were anaesthetized intraperitoneally with pentobarbital sodium (30 mg/kg), the left sciatic nerve was exposed, and part (15 mm long) of which was removed. The defected parts of the sciatic nerve of the nude rats were bridged with silicone tubes each containing 1×10⁵ AC133 positive cells embedded in atelocollagen (FIG. 4). As a control group, bridging was conducted with a silicone tube filled with atelocollagen alone on another group of rats. Eight weeks into transplantation, visual observation, histological assessment, and electrophysiological assessment were conducted, and wet weights of muscles were measured on each group.

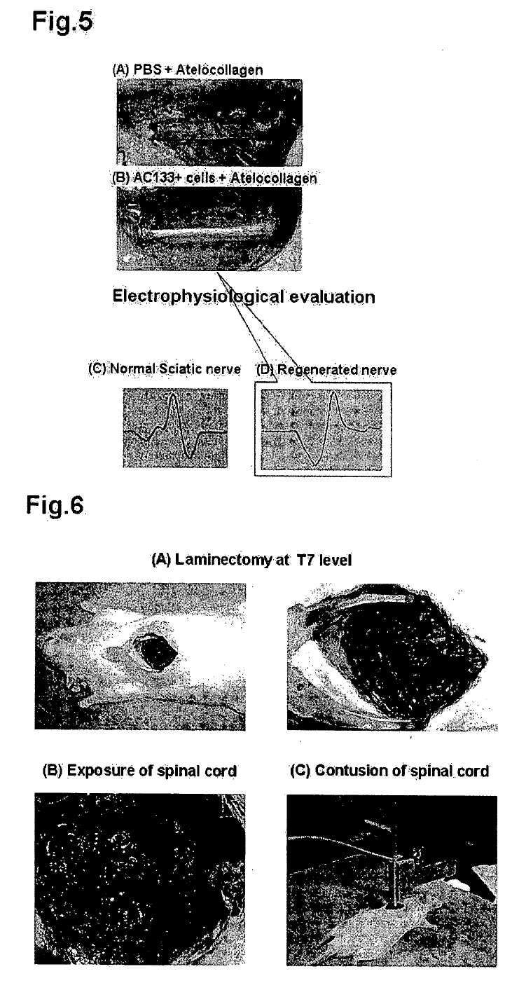

The results are shown in FIG. 5. The bridging of neural tissue in the tube was visually observed in all samples in the AC133 positive cell group, but no bridging was observed in the control group (FIGS. 5A, B). Satisfactory axonal regeneration was histologically observed in the AC133 positive cell group. In electrophysiological assessment, a muscular evoked potential equivalent to normal potential could be induced in all the samples in the AC133 positive cell group (FIG. 5D), but was not induced in any sample in the control group. In the wet weight of the anterior tibial muscle, there was no difference in amyotrophy between the experimental group and the control group.

These results demonstrate that the peripheral blood-derived AC133 positive cells can physically and functionally regenerate the injured peripheral nerve (sciatic nerve).

### EXAMPLE 3

In this example, AC133 positive cells were intravenously administered, and accumulation of the cells in an injured spinal cord and regenerative effect thereof on the injured spinal cord were investigated.

Spinal Cord Injury

Adult male athymic nude rats (weighing 230-250 g, F344/N Jcl rnu/rnu, CLEA, Japan, Inc, Tokyo, Japan) were anesthetized with pentobarbital sodium (50 mg/kg, intraperitoneally), and laminectomy was performed microscopically at the T7 level of the spinal cord. A 25 g rod was placed on the spinal cord for 90 seconds to induce a contusion lesion. The wound were sutured in multiple layers.

Transplantation

We used G-CSF mobilized human peripheral blood derived AC133 positive cells (Cambrex) for transplantation. We transplanted 100 μl of phosphate-buffered saline (PBS) alone in control group and 1×10⁵ AC133 positive cells in 100 μl of PBS in experimental group by a single intravenous injection via the femoral vein immediately after the injury. All rats had free access to food and water throughout the study. Manual urination management was carried out twice a day until the bladder function was restored.

Behavioral Assessment

Hind-limb motor function was scored with the BBB locomotor rating scale using an open field environment on days 1-7 and then every week up to the sixth week (n=6 in each group). Rats were recorded on video and two examiners without surgeon followed a mark.

Tissue Harvest

Rats were deeply anesthetized by injection of pentobarbital sodium (100 mg/kg), and perfused transcardially with 100 ml PBS followed by 100 ml cold 4% paraformaldehyde (PFA), pH 7.4. The spinal cord tissues at the lesion site (4 mm long) were carefully resected and invested in analysis for real time PCR or frozen by liquid nitrogen for immunohistochemistry.

The Area of Cavity

For the measurement of the area of cavity, the rats in both group were harvested (n=4 in each group) at 3 weeks after transplantation and the frozen spinal cord tissue was cut into axial sections in a cryostat at 10 μm thickness. The sections were observed by fluorescene microscope (Leica Microsystems, AG, Germany) without staining. The cavity was measured by using Scion Image computer analysis software (Scion Corporation, Frederick, Md., USA).

Immunohistochemistry

The ideal frozen sections (6 μm) were immunologically stained using anti-Human Nuclear Antigen (HNA) antibody (1:100, Chemicon) and anti-human mitochondria (hMit) antibody (1:200, Chemicon) for detection of intravenously administrated AC133 positive cells, anti-Isolectin B4 antibody (1:100, Vector) and von Willebrand Factor (vWF) (1:50, Santa Cruz) for detection of angiogenesis, anti-neurofilament (NF) antibody (1:100, Chemicon) for detection of survived axons, anti-Gap43 antibody (1:1000, Chemicon) for detection of regenerated axons, and anti-CXCR4 antibody (1:500, Anaspec) for detection of neural progenitor cells. The sections were cut into sagittal or axial sections and fixed with 2% PFA for 10 minutes at 4° C., additionally fixed with 4% PFA for 5 minutes at 4° C., washed with cold PBS 3 times for 3 minutes each, permeated with 0.1% Triton X-100 in PBS for 30 minutes at room temperature, blocked with 0.1% Triton X-100 in 10% goat for 1 hour at room temperature, and then reacted with primary antibodies as above overnight at 4° C. On the following day, the tissues were washed with PBS 2 times for 3 minutes each, incubated for 2 hour in the dark with Alexa Fluor 488 anti-rabbit or mouse IgG (1:400) and Alexa Fluor 568 anti-rabbit or mouse IgG (1:400) at room temperature. The immunostained cells were washed several times, counterstained with 4′,6′-diamidino-2-phenylindole (DAPI; Vector, Burlingame, Calif., USA), and observed under a fluorescence microscope.

Statistical Analysis

BBB locomotor rating scale scores were analyzed with repeated-measures analysis of variance at all time points and with the Mann-Whitney U-test at each time point. The area of cavity, the number of CXCR4 positive cells, and mRNA expression levels of various factors were analyzed with the Mann-Whitney U-test. Results were expressed as mean ± the standard error of the mean. Significance was set at p<0.05.

Results

Recovery of Motor Function

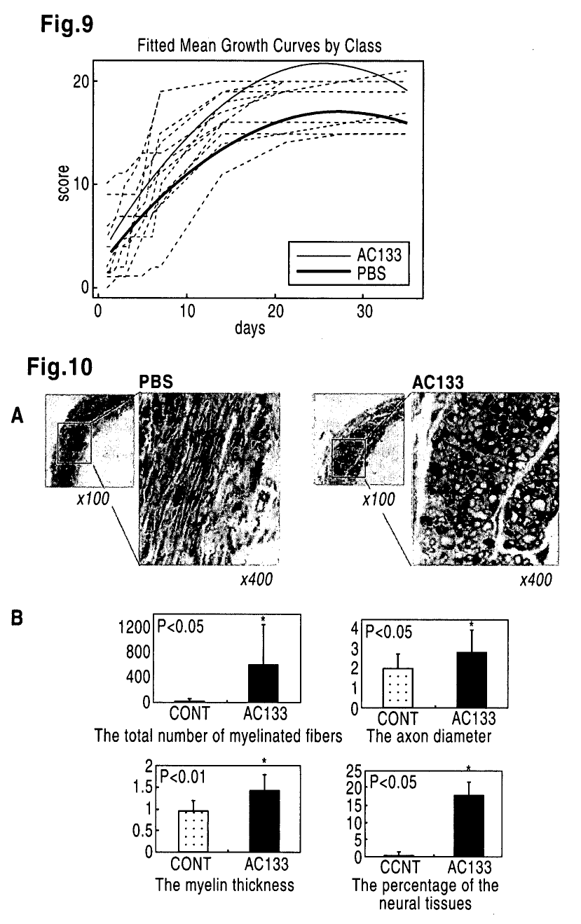

The hind-limb motor function scored with the BBB scale showed improvement in both groups. There were not significant differences of the BBB score between the two groups within 6 days after injury. However, after 1 week, the BBB score of experimental group demonstrated significant improvement compared to the control group at every week up to sixth week. At 6 weeks after injury, the BBB score of experimental group was 20.6±0.4 and that of control group was 16.3±1.2, showing significant difference between them (p<0.01) (FIG. 9).

Measurement of the Area of Cavity

The area of cavity of injured spinal cord in axial section at 3 weeks after injury was significantly smaller in experimental group (0.21±0.06 mm2) than that in control group (0.85±0.18 mm2) (p<0.05) (FIG. 8).

Immunohistochemistry

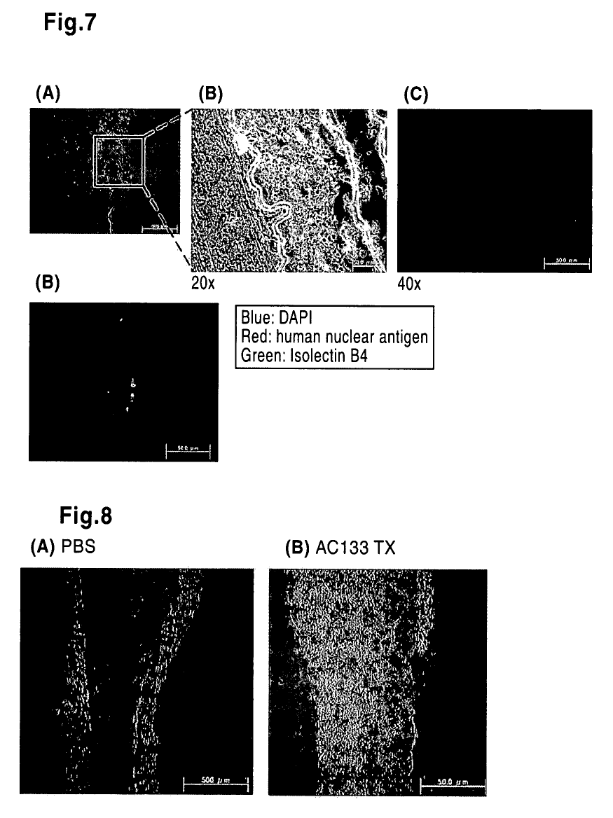

At 1 day after injury, there were HNA positive cells and isolectin B4 positive cells on the surface of the injured spinal cord (sagittal section) of rats in experimental group (FIG. 7). It was recognized that intravenously administered AC133 positive cells migrated into the injured spinal cord and were taken in vascular endothelial cells. Immunofluorescent stain was conduced on the AC133 positive cell transplanted group and control group 3 days into the transplantation in the experiment using an injured spinal cord model. At 3 days after injury, double staining of NF and Gap43 (sagittal section) showed that the expression of Gap43 around the injured axons was much more in experimental group than that in control group (data not shown). At 3 days after injury, the number of CXCR4 positive cells (axial section) in experimental group was 32.0±2.8 and that in control group was 9.3±1.3 showing significant difference between them (n=4 in each group) (p<0.05). At 1 week after injury, triple staining of vWF, DAPI, and hMit (axial section) showed that intravenously administered AC133 positive cells still remained in vascular endothelial cells and revascularization was confirmed (data not shown).

### EXAMPLE 4

### Morphometric Evaluations

The regenerated tissues that formed in the tube in Example 2 were collected and pre-fixed with 2.5% glutaraldehyde, postfixed with % osmic acid, and embedded in Epon according to a standard procedure and cut cross-sectionally by 0.5 μm thick. Each section was stained with 0.5% (w/v) toluidine blue solution and examined by optical microscopy.

Digitized image were imported into a personal computer using Photoshop software (Adobe Systems Inc., San Jose, Calif., USA). Computer analysis of this digitized information based on gray and white scales was used to measure the total number of fibers and the total fascicular area in sections of the mid point of the tube using the image analysis software NIH Image program (the National Institutes of Health, Bethesda, Md., USA). Six randomly selected fields per nerve were evaluated for myelin thickness and axon diameter at 100× magnification. Based on these data, additional calculations of the total number of myelinated fibers, and percentage of neural tissue (100×(neural area)/(intrafascicular area)) were made. An observer blinded to the experimental groups made all measurements.

The toluidine blue staining revealed that the atelocollagen gel remained at the center of the tube and tissues were newly formed around the atelocollagen gel (FIG. 10A). In particular, regeneration of medullated nerve significant both in number and quality was observed in the AC133 positive cell group.

The statistical analysis showed significant differences between the control group and the AC133 positive cell group in all the points of assessment, i.e., number of myelinated fibers, axon diameter, myelin thickness, and percentage of neural tissues (FIG. 10B).

### EXAMPLE 5

### Immunofluorescent Staining

The regenerated tissues embedded in Example 4 were sectioned, and 6-μm serial sections were mounted on silane-coated glass slides and air-dried, followed by being fixed with 4.0% paraformaldehyde at 4° C. for 5 minutes and stained immediately. To evaluate axonal regeneration and detect transplanted human cells in the regenerated tissues histologically, immunohistochemistry was performed with the following antibodies; S-100 protein (Chemicon International, Inc, Temecula, Calif.) to detect Schwann cells, human nuclear antibody (HNA) (Chemicon International, Inc, Temecula, Calif.) to detect transplanted human cells, and von Willebrand factor (vWF) protein (Santa Cruz Biotechnology, Santa Cruz, Calif.) to detect endothelial cells. The secondary antibodies for each immunostaining were as follows: Alexa Fluor 488 or 568-conjugated goat anti-mouse IgG1 (Molecular Probes) for HNA, Alexa Fluor 488 or 568-conjugated goat anti-rabbit IgG (Molecular Probes) for S-100 and vWF protein. A 4,6-diamidino-2-phenylindole (DAPI) solution was applied for 5 minutes for nuclear staining (data not shown).

The immunofluorescent staining of regenerated tissues derived from the AC133 positive cell group at 8 weeks after transplantation demonstrate the expression of S-100 protein, marker of Schwann cells, in the regenerated tissues derived from the AC133 positive cell group at 8 weeks after transplantation. In addition, there were a lot of double positive cells with HNA (data not shown). These results demonstrate that the transplanted human peripheral blood-derived AC133 positive cells differentiated into Schwann cells. With vWF staining, although vasculatures were observed in the regenerated axons, there is no double positive cell with vWF and HNA. Differentiation into vascular endothelial progenitor cells was not confirmed in transplanted human peripheral blood-derived AC133 positive cells (data not shown). In contrast in control group, only scar formations were observed (data not shown).

### EXAMPLE 6

### Reverse Transcriptase-Polymerase Chain Reaction (RT-PCR) Analysis of RNA Isolated from Regenerated Tissue

Total RNA was obtained from regenerated tissues in silicone tubes at 8 weeks in Example 2 and freshly isolated peripheral blood AC133 positive cells using the Qiagen RNA isolation kit (Qiagen KK, Tokyo, Japan) according to the manufacturer's procedure. The first-strand cDNA was synthesized using the RNA LA PCR Kit version 1.1 (Takara, Otsu, Japan), and amplified by Taq DNA polymerase for RT-PCR analysis (AmpliTaq DNA polymerase, Applied Biosystems) using gene-specific primer sets a's described below. PCR was performed with a thermal cycler (MJ mini Gradient Thermal Cycler, Bio-Rad Laboratories) under the following condition: 35 cycles of initial denaturation at 94° C. for 30 seconds, annealing at 56° C. for 1 minute, and extension at 72° C. for 30 seconds. All procedures were followed manufacturer's instructions. The RT-PCR products were electrophoresed in 2% agarose gel containing ethidium bromide in tris-borate-EDTA electrophoresis buffer; visualized by ultraviolet transillumination. As a control, we used contralateral sciatic nerves for axon-related genes.

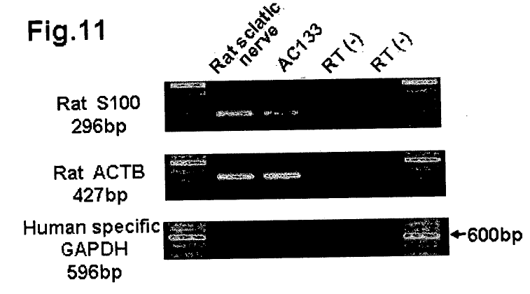

The results are shown in FIG. 11. Regenerated tissues in the human peripheral blood-derived AC133 positive cell transplanted group expressed S-100 protein and human-specific GAPDH. These results demonstrate that Schwann cell marker was expressed at genetic level and transplanted cells were survived in the regenerated tissues in the AC133 positive cell transplanted group.

Primers used in the experiments will be described below.

For characterization of the isolated AC133 positive cells, following primers were designed:

AC133 primer sequence (365 bp):

| | |
|---|---|
| SEQ ID NO 1: | |
| (sense) | 5′-CGTGGATGCAGAACTTGACAAC-3′ |
| SEQ ID NO 2: | |
| (anti-sense) | 5′-CACACAGTAAGCCCAGGTAGTAAAA-3′ |

As neuron and axon-related genes, we designed primers detecting human MAP2 and S-100 protein sequences:

hMAP2 (371 bp):

| | |
|---|---|
| SEQ ID NO 3: | |
| (sense) | 5′-GATGGCTTCAGGGCTAAACA-3′ |
| SEQ ID NO 4: | |
| (anti-sense) | 5′-CAGCAGGTGGGCAAGGTAT-3′ |

hS-100 protein sequence (431 bp):

| | |
|---|---|
| SEQ ID NO 5: | |
| (sense) | 5′-TGGAGACGGCGATGGAG-3′ |
| SEQ ID NO 6: | |
| (anti-sense) | 5′-CAGGCTTGGACCGCTACTCT-3′ |

To detect endothelial phenotypes, we used primers for human vascular endothelial cadherin (VE-cadherin) and vascular endothelial growth factor receptor type 2 (VEGFR2/KDR)

hVE-cadherin sequence (461 bp):

| | |
|---|---|
| SEQ ID NO 7: | |
| (sense) | 5′-ACGCCTCTGTCATGTACCAAATCCT-3′ |
| SEQ ID NO 8: | |
| (anti-sense) | 5′-GGCCTCGACGATGAAGCTGTATT-3′ |

hVEGFR2/KDR sequence (468 bp):

| | |
|---|---|
| SEQ ID NO 9: | |
| (sense) | 5′-CAAATGTGAAGCGGTCAACAAAGTC-3′ |
| SEQ ID NO 10: | |
| (anti-sense) | 5′-ATGCTTTCCCCAATACTTGTCGTCT-3′ |

To evaluate regenerated tissues from nude rats, we used primer sequences designed for rat sequences as follows:

rS-100 protein sequence (296 bp):

| | |
|---|---|
| SEQ ID NO 11: | |
| (sense) | 5′-GGAAGGGGACAAATATAAGC-3′ |
| SEQ ID NO 12: | |
| (anti-sense) | 5′-GGCAAGGATGGGTACATAG-3′ |

As internal control, we used beta-actin sequence (427 bp):

| | |
|---|---|
| SEQ ID NO 13: | |
| (sense) | 5′-ACCCTAAGGCCAACCGTGAAA-3′ |
| SEQ ID NO 14: | |
| (anti-sense) | 5′-TCATTGCCGATAGTGATGACCTGAC-3′ |

In this study we applied human-specific primers to confirm engraftment of transplanted human cells at transcriptome level. To avoid interspecies cross-reactivity of the primer pairs between human and rat genes, we designed the human-specific GAPDH primer sequence (596 bp) (hGAPDH):

| | |
|---|---|
| SEQ ID NO 15: | |
| (sense) | 5′-CTGATGCCCCCATGTTCGTC-3′ |
| SEQ ID NO 16: | |
| (anti-sense) | 5′-CACCCTGTTGCTGTAGCCAAATTCG-3′ |

All primers used in the present study were designed using Oligo software (Takara).

Sequence Listing

What is claimed is:

1. A therapeutic agent for a neuropathy selected from the group consisting of a spinal cord injury, a sciatic nerve injury, and a neurotmesis, comprising AC133 positive cells.

2. The therapeutic agent according to claim 1, wherein the AC133 positive cells are derived from peripheral blood, cord blood, placental blood, or a bone marrow.

3. The therapeutic agent according to claim 2, wherein the AC133 positive cells are derived from peripheral blood.

4. The therapeutic agent according to claim 3, wherein the AC133 positive cells are of human origin.

5. The therapeutic agent according to any one of claims 2 to 4, wherein the neuropathy is a spinal cord injury.

6. The therapeutic agent according to any one of claims 2 to 4, wherein the neuropathy is a sciatic nerve injury.

7. The therapeutic agent according to any one of claims 2 to 4, wherein the neuropathy is a neurotmesis.

8. The therapeutic agent according to claim 6, wherein the sciatic nerve injury is an entrapment neuropathy.
